(19) United States
(12) Plant Patent Application Publication
(10) Pub. No.: US 2007/0180587 P1
(43) Pub. Date: Aug. 2, 2007

(54) Botanical designation

(76) Inventor: Mogens N. Olesen, Fredensborg (DK)

Correspondence Address: POULSEN ROSER APS, 620 S. FRONT STREET, CENTRAL POINT, OR, 97502, US

(21) Appl. No.: 11/345,876

(22) Filed: Feb. 1, 2006

(51) Int. Cl. A01H 5/00

(52) U.S. Cl. PLT/118

(57) ABSTRACT

A new miniature rose plant that has abundant, yellow-orange flowers and attractive foliage. The variety successfully propagates from softwood cuttings and is suitable for year-round production in commercial glasshouses. This new and distinct variety has shown to be uniform and stable in the resulting generations from asexual propagation.

### BOTANICAL DESIGNATION

Rosa hybrid

### VARIETY DENOMINATION

‘Poulpai001’

### SUMMARY OF THE INVENTION

The present invention constitutes a new and distinct variety of miniature rose plant which originated from a controlled crossing between the female seed parent, an unnamed seedling, and the male pollen parent ‘Poulhi008’, described and illustrated U.S. Plant Pat. No 15,722.

The two parents were crossed during the summer of 2002 and the resulting seeds were planted in a controlled environment in Fredensborg, Denmark. The new variety, named ‘Poulpai001’, originated as a single seedling from the stated cross.

The new variety may be distinguished from its female seed parent by the following combination of characteristics:
1. The seed parent is taller in growth height than ‘Poulpai001’.
2. The seed parent has lighter yellow flower coloration than ‘Poulpai001’.

The new variety may be distinguished from its male pollen parent by the following combination of characteristics:
1. The pollen parent has pink flowers, while ‘Poulpai001’ has yellow-orange flowers.
2. The pollen parent has very glossy leaves, while ‘Poulpai001’ has matte leaves.

The objective of the hybridization of this rose variety for commercial culture was to create a new and distinct variety with unique qualities, such as:
1. Uniform and abundant yellow-orange flowers with old fashion flower form;
2. Vigorous and compact growth;
3. Year-round flowering under glasshouse conditions;
4. Suitability for production from softwood cuttings in pots;
5. Durable flowers and foliage which make a variety suitable for distribution in the floral industry.

This combination of qualities is not present in previously available commercial cultivars of this type, known to the inventors, and distinguish ‘Poulpai001’ from all other varieties of which we are aware.

As part of the rose development program, Mogens N. Olesen germinated the seeds from the aforementioned hybridization and conducted evaluations on the resulting seedlings in a controlled environment in Fredensborg, Denmark. ‘Poulpai001’ was selected by the inventor as a single plant from the progeny of the hybridization in 2002.

Asexual reproduction of ‘Poulpai001’ by vegetative cuttings and traditional budding was first done by Mogens N. Olesen in the nursery in Fredensborg, Denmark in 2003. This initial and other subsequent propagations conducted in controlled environments have demonstrated that the characteristics of ‘Poulpai001’ are true to type and are transmitted from one generation to the next.

### BRIEF DESCRIPTION OF THE DRAWING

The accompanying color illustration shows as true as is reasonably possible to obtain in color photographs of this type, the typical characteristics of the buds, flowers, leaves, and stems of ‘Poulpai001’. Specifically illustrated in the drawing:

FIG. 1.1; Flower bud and flowers at various stages of opening;

FIG. 1.2; Flower petals, detached;

FIG. 1.3; Pedicel, receptacle, sepals, and reproductive flower parts;

FIG. 2.1; Juvenile growth;

FIG. 2.2; Intermediate juvenile leaves;

FIG. 2.3; Mature leaves;

FIG. 2.4; Bare stem exhibiting prickles;

FIG. 3.0; Open flowers showing flower form and progression of flower color changes as flowers mature; and

FIG. 4.0; Open flowers and leaves.

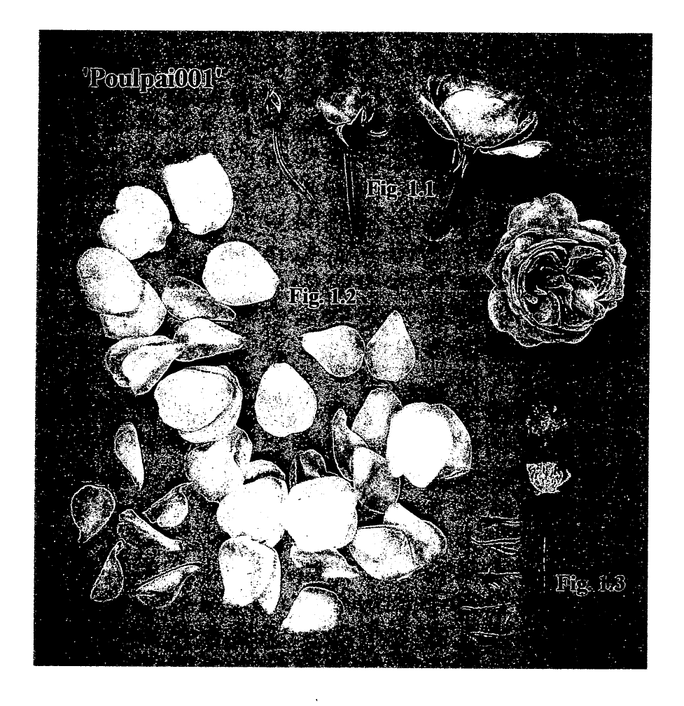

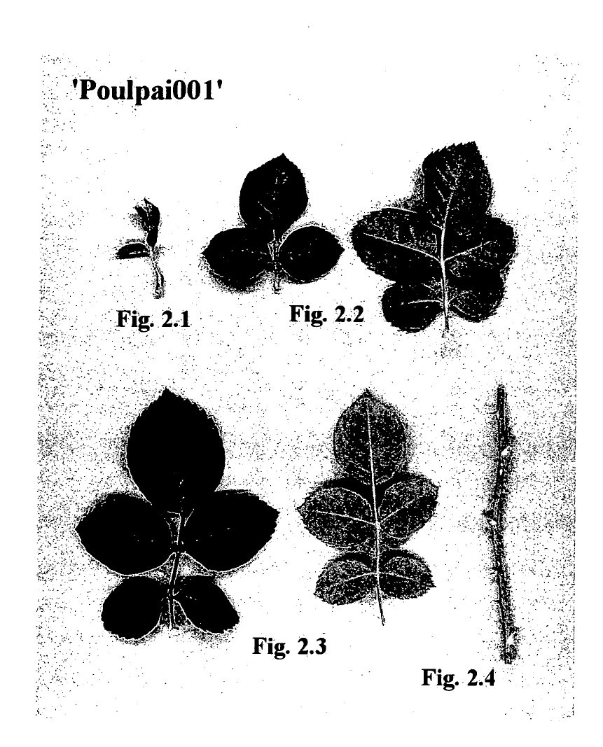

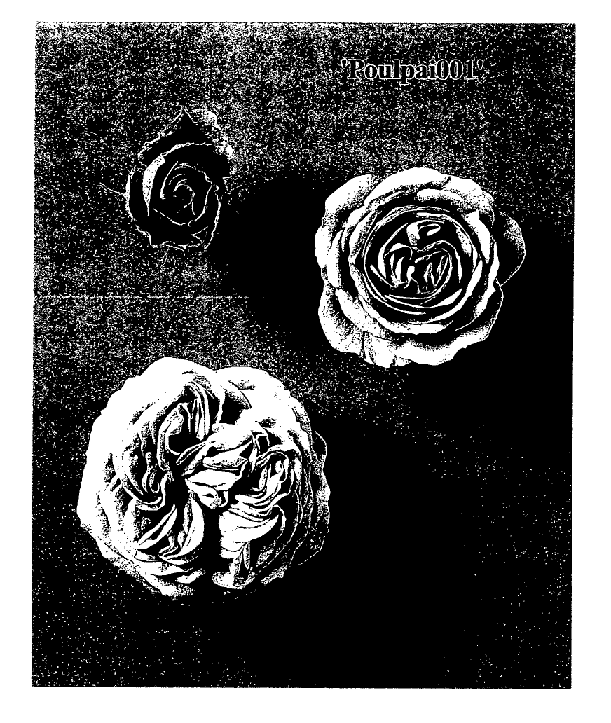

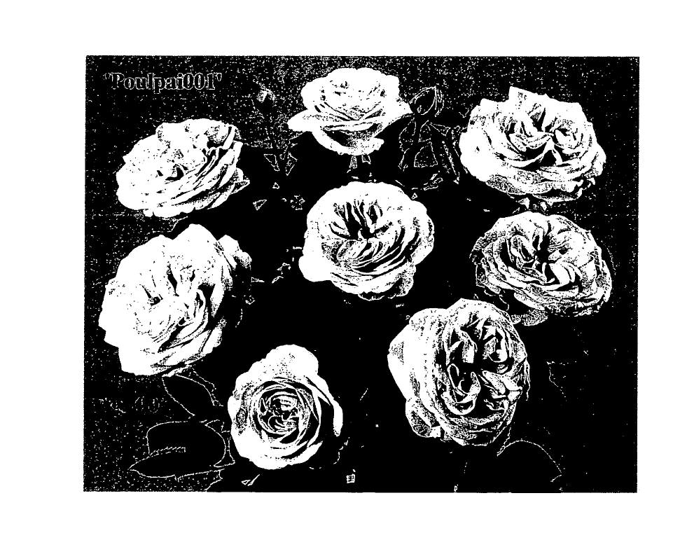

### DETAILED DESCRIPTION OF THE VARIETY

The following is a description of ‘Poulpai001’, as observed in its growth in Burlington, Ontario. Observed plants are 14 weeks of age and were cultivated in 15 cm pots under normal glasshouse conditions. Color references are made using The Royal Horticultural Society (London, England) Colour Chart, 2001, except where common terms of color are used.

For a comparison, several physical characteristics of the rose variety ‘Poulpah022’, U.S. Plant Patent Application No. 10/961,001 dated Oct. 7, 2004 are compared to ‘Poulpai001’ in Chart 1.

CHART 1

| | ‘Poulpai001’ | ‘Poulpah022’ |
|---|---|---|
| Petal count; | 50 to 55 petals, 1 to 5 of | 50, 7 to 10 of which are |
| | which are petaloids | petaloids |
| Flower Depth: | 35 to 40 mm | 35 mm |
| General Tonality | Yellow-Orange Group 23A | Yellow Group 13D to |
| of Flower Color: | towards the flower center. | Yellow-Orange Group |
| | The periphery is Yellow- | 16B |
| | Orange Group 16A to | |
| | Yellow Group 10A. | |

### FLOWER AND FLOWER BUD

Blooming habit: Recurrent.
Flower bud:
   Size.—Upon opening, 20 to 24 mm in length from base of receptacle to end of bud. 11 to 13 mm in diameter.
   Bud form.—Pointed ovate.
   Bud color.—As sepals unfold, petals are Yellow-Orange.Group 22A to Orange Group 24B with intonations of Yellow Group 10A. Additional pigments the color of Yellow-Green Group 144B to 145A commonly observed.
   Sepals.—Upper Surface: Color: Yellow-Green Group 147B. Texture: Smooth with a medium amount of pubescence observed. Lower Surface: Color: Yellow Green Group 146B. Texture: Smooth.
   Shape.—Apex: Cirrhose to acuminate. Base: Flat at union with receptacle. Margins: Margins have weak foliaceous appendages on three of the five sepals. Size: On average, sepals are 20 mm long by 5 to 6 mm wide.
   Receptacle.—Surface Texture: Smooth. Shape: Urn-shaped. Size: Normally 6 mm in height by 6 to 7 mm wide. Color: Yellow Green Group 144A.
   Pedicel.—Surface: Slightly rough due to the presence of stipitate glands. Length: 40 to 60 mm. Diameter: 3 mm average. Color: Yellow-Green Group 144 A. Strength: Medium strength.
   Borne.—Normally singly.
Flower bloom.—
   Fragrance.—Moderate wild rose to vegetal scent.
   Duration.—As a pot plant, flowers last from 21 to 28 days.
   Size.—Diameter of open flowers is 60 to 65 mm, while the depth is normally 34 to 40 mm.
   Form.—General shape is a cupped and quartered rosette with many petals packed tightly into quarter sections.
   Shape of flower, side view.—Upon opening, the upper portion is flat while the lower portion is generally concave. After opening the upper portion is flat while the lower portion is generally concave.
   Petalage.—There are normally 50 to 55 petals, 1 to 5 of which are underdeveloped petaloids.
Color:
   Upon opening, petals.—Outermost petals: Upper Surface: Yellow Group 11 A with faint intonations of Yellow-Orange Group 16A. Lower Surface: Yellow-Orange Group 16B with an overlay of Orange Group 26B. Innermost petals: Upper Surface: Yellow-Orange Group 15A with a faint overlay the color of Yellow Group 13B. Lower Surface: Yellow-Orange Group 16B with an overlay the color of Orange Group 29A to Orange Group 26B.
   Upon opening, basal petal spots.—Upper Surface: Yellow Group 13B. Lower Surface: Yellow-Orange Group 15D.
   After opening, petals.—Outermost petals: Upper Surface: Yellow-Orange Group 14C. Lower Surface: Yellow-Orange Group 14C with an overlay the color of Yellow-Orange Group 16B. Innermost petals: Upper Surface: Yellow-Orange Group 15C to Yellow Group 12C. Lower Surface: Yellow-Orange Group 16B with intonations of Orange Group 29A to Orange Group 26B.
   After opening, basal petal spots.—Upper Surface: Yellow Group 13B. Lower Surface: Yellow-Orange Group 15D.
General Tonality: On open flower Yellow-Orange Group 23A towards the flower center. The periphery is Yellow-Orange Group 16A to Yellow Group 10A. Flower color progresses from orange to orange-yellow to yellow as blooms reach full maturity.
Petals:
   Petal reflex.—Outer petals are reflexed, while the inner petals are not reflexed.
   Petal margin.—Entire and uniform. Occasionally petals exhibit a cleft at the center of the apex.
   Shape.—Generally varying from broad to narrow elliptical. Base: Acute. Apex: Rounded.
   Size.—Outer petals are larger, 40 mm in length by 40 mm wide. Inner petals are 30 mm in length by 20 mm wide.
   Thickness.—Average.
   Texture.—Smooth.
Petaloids:
   Quantity.—Normally, there are 1 to 5.
   Size.—15 mm long by 8 mm wide.
   Shape.—Apex is rounded. Base is acute. Generally the petaloids are narrow elliptic and somewhat asymmetric.
   Petaloid Color.—Upper Surface: Yellow-Orange Group 15A with a faint overlay the color of Yellow Group 13B. Lower Surface: Yellow-Orange Group 16B with an overlay the color of Orange Group 29A to Orange Group 26B.
Reproductive organs:
   Pollen.—Pollen not observed.
   Anthers.—Size: Normally 2 mm long. Color: Yellow Group 13C with margins the color of Greyed-Orange Group N163A. Quantity: Normally 50 to 55.
   Filaments.—Color: Yellow 13C. Length: 7 to 8 mm in length.
   Pistils.—Length: 11 to 12 mm long. Quantity: 45 to 50.
   Stigmas.—Superior in position, relative to the length of the filaments and the height of the anthers. Color: Greyed-Yellow Group 160B to Yellow-Green Group 151D.
   Styles.—Color: Green-White Group 157D to Yellow-Green Group 154D.
   Seed formation.—Not observed.

### PLANT

Plant growth.—Compact and upright, narrowly bushy. Grown in 15cm pots, the plants generally reach a height of 35 to 40 cm. Width is normally 25 to 35 cm.
Stems:
   Color.—Young wood: Yellow-Green Group 146B. Older wood: Yellow-Green Group 146B.
   Internodal distance.—25 cm on average.
   Length of stems.—On average, canes are 35 cm from the base of the stem to the flowering portion.
   Stem diameter.—Normally 3 mm.
   Surface texture.—Young wood: Smooth. Older wood: Smooth.
Prickles: Incidence: Normally 8 per 10 cm of stem. Size: Average length is 5 mm. Color: Juvenile prickles are Greyed-Red Group 179C. Shape: Upper portion is linear, while the lower portion is concave.
Plant foliage: Normally 5 leaflets on normal leaves in middle of the stem.
   Compound leaf size.—95 to 100 mm (l) by 60 to 85 mm (w).
   Quantity.—On average there are 4 leaves per 10 cm of stem.
   Leaf color.—
   Juvenile foliage.—Upper Leaf Surface: Yellow-Green Group 146B. Lower Leaf Surface: Yellow-Green Group 146D. Anthocyanin: Greyed-Red Group 182A on the lower surface of leaflets, and the upper margins of leaflets. Mature foliage: Upper Leaf Surface: Yellow-Green Group 147A to Green Group 139A. Lower Surface: Yellow-Green Group 147C with anthocyanic intonations on some of the mature leaflets, Greyed-Red Group 182A in color.
Plant leaves and leaflets:
   Stipules.—Size: 11 to 23 mm in length. Shape: Linear, slightly broad based with outward extending apecies. Margins: Finely serrated with few stipitate glands. Color: Yellow-Green Group 146A.
   Petiole.—Length: 12 to 20 mm. Diameter is normally 1 to 1.5 mm. Color: The upper portion is Yellow-Green Group 146B with occurrences of anthocyanin the color of Greyed-Purple Group 183C. The lower surface is Yellow-Green Group 146C in color. Underneath: Small prickles observed.
   Rachis.—Size: 35 to 40 mm in length. Upper Surface: Color is Yellow-Green Group 146B. Anthocyanic pigments the color of Greyed-Purple Group 183C observed. Lower Surface: Color is Yellow-Green Group 146C. Small prickles observed.
   Leaflet.—Size: 40 to 52 mm in length by 28 to 39 mm wide. Margin: Deeply serrated. General Shape: Elliptical. Apex Shape: Cuspidate. Base Shape: Obtuse to round. Texture: Smooth. Arrangement: Odd pinnate. Venation: Reticulate. Leaf Gloss: Matte finish.
Disease resistance:

Somewhat above average resistance to powdery and downy mildew, black spot, and Botrytis under normal controlled growing conditions.

1. A new and distinct variety of rose plant of the miniature class named ‘Poulpai001’, substantially as illustrated and described herein, due to its abundant, yellow-orange flowers, vigorous growth, compact habit, suitability for production from softwood cuttings in pots, and durable flowers and foliage that make the variety suitable for distribution in the floral industry.
